(19) United States
(12) Patent Application Publication
(10) Pub. No.: US 2020/0017603 A1
(43) Pub. Date: Jan. 16, 2020

(54) NOVEL USES OF PCSK9 INHIBITORS AND RELATED MEDICATIONS

(71) Applicant: BHL PATENT HOLDINGS, LLC, TOWANDA, PA (US)

(72) Inventor: BRUCE H. LEVIN, PHILADELPHIA, PA (US)

(21) Appl. No.: 16/465,583

(22) PCT Filed: Dec. 1, 2017
(86) PCT No.: PCT/US2017/064203

§ 371 Date: May 31, 2019

Related U.S. Application Data
(60) Provisional application No. 62/428,641, filed on Dec. 1, 2016.

(51) Int. Cl. C07K 16/40; A61P 1/16; A61P 3/06; A61K 31/20; A61K 31/397; A61K 31/202

(52) U.S. Cl. CPC C07K 16/40; A61P 1/16; A61P 3/06; A61K 2039/505; A61K 31/397; A61K 31/202; C07K 2317/76; A61K 31/20

(57) ABSTRACT

A method comprising administering an agent, for example a PCSK9 inhibitor, to a patient who suffers from fatty liver disease or is at risk of fatty liver disease, thereby reducing the risk of fatty liver disease, severity of fatty liver disease, symptoms of such disease, or signs of such disease and a method comprising administering a PCSK9 inhibitor to a patient having symptoms of elevated liver enzymes to ameliorate said symptoms.

### CROSS-REFERENCE TO RELATED APPLICATIONS

Benefit of U.S. provisional application No. 62/428,641, entitled “METHOD OF TREATING FATTY LIVER DISEASE AND OTHER PATHOLOGIES, filed Dec. 1, 2016, and International Application No. PCT/US17/64203, entitled NOVEL USES OF PCSK9 INHIBITORS AND RELATED MEDICATIONS, tiled Dec. 1, 2017 is claimed, and are hereby incorporated by reference.

### BACKGROUND OF THE INVENTION

Proprotein convertase subtilisin/kexin type 9 (hereafter “PCSK9”) inhibitors are used to lower cholesterol levels in adult patients with familial hypercholesterolemia or ASHD who are on the highest tolerated statin doses along with diet to provide additional lowering of LDL cholesterol. The first two PCSK9 inhibitors, alirocumab (trade name Praluent) and evolocumab (trade name Repatha), were approved as once every two week injections, by the U.S. Food and Drug Administration in 2015 for lowering LDL-particle concentrations when statins and other drugs were not sufficiently effective or poorly tolerated.

PCSK9 inhibitors have not previously suggested or used for improving liver health, status and function and decrease levels of liver enzymes which may be elevated during sub clinical or clinical liver pathologies. Nor have said inhibitors been suggested for decreasing inflammation in joints, eyes, or other areas subject to inflammation.

### SUMMARY OF THE INVENTION

The present invention comprises administering a PCSK9 inhibitor to a patient at risk of fatty liver disease or who suffers from symptoms of such disease, thereby reducing the risk of said disease, severity of said disease, symptoms of said disease, and signs of said disease. The inhibitor can be administered to a patient suffering from any type of liver disease.

The invention also comprises administering such an inhibitor to decrease inflammation of joints, eyes, or other areas of inflammation.

Utilizing statin agents alone may not be adequately effective and can increase liver damage and likelihood of developing diabetic pathologies. The invention comprises administering a statin agent in combination with a PCSK9 inhibitor.

Variants of PCSK9 can reduce or increase circulating cholesterol. LDL-particles are removed from the blood when they bind to MLR on the surface of cells and are taken inside the cells. When PCSK9 binds to an LDLR, the receptor is destroyed along with the LDL particle. PCSK9 degrades LDLR by preventing the hairpin conformational change of LDLR. If PCSK9 does not bind, the receptor will return to the surface of the cell and can continue to remove LDL-particles from the bloodstream.

In another aspect the invention comprises administering a PCSK9 inhibitor to a patient having elevated liver enzymes to reduce said enzymes.

In yet another aspect, the invention comprises method comprising administering a PCSK9 inhibitor to a patient having symptoms or signs of hepatic dysfunction to ameliorate said symptoms or signs and improve hepatic function.

The invention also comprises administering a PCSK9 inhibitor to a patient suffering from inflammation to ameliorate or decrease said inflammation.

The invention further comprises administering to a patient an agent other than a PCSK9 inhibitor to decrease lipid, fatty acid, or cholesterol synthesis, or to increase clearance of lipid, fatty acid, or cholesterol, in order to decrease risk of fatty liver disease, and decrease symptoms or signs of inflammation.

### DETAILED DESCRIPTION OF THE INVENTION

According to this invention , PCSK9 inhibitors decrease metabolic, oxidative, free radical, lipogenic or other stresses to the liver and thereby improve liver health, status and/or function.

PCSK9 inhibitors effectively decrease stress on the liver and decrease liver damage and ameliorate liver pathology and manifestations thereof.

PCSK9 inhibitors are particularly useful for treatment of fatty liver disease, or other insults following tetracycline, alcohol, antibiotic or other drug, toxin or chemical exposures which result in hepaticyte vacuolization or fatty liver.

In patients who cannot tolerate statins, PCSK9 inhibitor monotherapy can be employed.

Areas of particular theoretical concern for chronic administration of alirocumab (brand name PRALUENT®) were identified based on the very low plasma cholesterol levels attainable with PCSK9 inhibitor therapy, especially with coadministration of statins. These included possible increases in bile acid concentrations in the intestine that could cause tumors, increased risk of HCV infection, modulation of cholesterol-derived hormones, immune suppression (in adults), and, similar to statins, risks for increased progression to type 2 diabetes and neurocognitive events. Overall, studies in animals administered alirocumab were reassuring regarding these theoretical concerns.

Theoretical risks have been identified with the PCSK9 inhibitors as a class. The following issues of potential (theoretical) concern have been identified:

Immunosuppression, especially when co-administered with HMG Co-A reductase inhibitors (statins). Immune cells (especially lymphocytes) are critically dependent on adequate membrane cholesterol concentrations. Co-administration of statins, which inhibit intracellular synthesis of cholesterol and are themselves immunomodulatory, could theoretically exacerbate the immunosuppressive potential of PCSK9 inhibitors.

Increased susceptibility to hepatitis C virus (HCV) infection: CD81, a critical component of the HCV receptor, is under negative regulation by PCSK9. Therefore, inhibition of PCSK9, by upregulating CD81 expression, might increase the availability of the HCV receptor, thereby increasing susceptibility to HCV infection.

Increased risk of colorectal cancer via increased intestinal bile acid load: Alirocumab, by increasing the expression of LDL-R, increases hepatic uptake of cholesterol. Given that the primary route of elimination of cholesterol by hepatocytes is conversion to bile acids, treatment with alirocumab may increase the load of bile acids delivered to the intestines, especially in hypercholesterolemic patients. Increased intestinal secondary bile acid load has been shown to increase intestinal cancer risk in rodents.

Hepatitis C virus (HCV) infectivity: A study by Labonte et al, identified regulation of CD81 cell-surface protein expression by PCSK9 as a potential pathway by which PCSK9 inhibitors might cause increased susceptibility to HCV infection and associated liver tumors. CD81 is a co-receptor for hepatitis C infection in humans. Labonte showed that expression of PCSK9, especially a modified non-secretable form, reduced CD81 and LDLR levels in immortalized human cells and provided resistance to HCV infection in vitro. PCSK9 inhibitors such as alirocumab may therefore increase CD81 expression resulting in greater infectivity of HCV.

Impaired liver regeneration: A concern for liver injury and alirocumab treatment comes from a published study conducted with the PCSK9 knockout mouse, When compared to littermates, PCSK9-null mice (but not PCSK9+/−mice) were markedly delayed in their ability to regenerate liver tissue following partial hepatectomy. Furthermore, the regenerating liver tissue exhibited necrotic foci. In these foci, the liver architecture was disrupted with swollen hepatocytes undergoing ballooning degeneration. Infiltration of red blood cells and leukocytes was also observed at the border of the necrotic areas. Whether this deficit is likely to be associated only with catastrophic liver injury (e.g., partial hepatectomy) or would also manifest following other liver injury (e.g., acetaminophen toxicity) is unknown. Of particular theoretical concern is the often transient, but sometimes severe liver injury induced by statins, which could theoretically be worsened by pharmacologically-induced loss of PCSK9 analogous to the PCSK9−/−mouse phenotype. However, liver toxicity was not exacerbated in a 3-month combination toxicity study with alirocumab when co-administered with atorvastatin in monkeys at doses that produced modest ALT increases and caused minimal to mild diffuse portal chronic inflammation, periductal chronic inflammation, and biliary ductular proliferation, but in the absence of severe toxicity. It is unknown whether recovery from more serious liver damage would be impacted by PCSK9 inhibitor therapies, including alirocumab.

One possible explanation for the failure of liver to properly regenerate in PCSK9 knockout mice owes to the discovery that HDL-C concentrations regulate bone marrow-derived endothelial progenitor cells, including the precursors of liver sinusoidal cells. Upon significant liver damage, liver regeneration is dependent upon endothelial progenitor cells to migrate from the bone marrow to the liver, where they are responsible for directing repair of damaged hepatic blood vessels and tissues. This process is required for proper liver repair after partial hepatectomy. It is tempting to speculate that low HDL-C impaired the production and migration of progenitor cells to direct liver regeneration in PCSK9−/−mice, Rats administered alirocumab had low HDL and sinusoidal cell defects were observed in the liver. This phenomenon was not observed in the monkey with alirocumab, even where HDL-C levels were greatly reduced. HDL-C was not reduced in humans administered alirocumab, which may argue that defects in liver regeneration observed in partially hepatectomized PCSK9 knockout mice have limited clinical relevance.

Immune modulation in adult animals: Inhibition of PCSK9 produces profound lowering of circulating cholesterol. The immune system is dependent on cholesterol for proper function. Clonal expansion of rapidly dividing immune cells (e.g., B-cells, T-cells, etc.) and cell-cell signaling are heavily dependent on cholesterol and cholesterol derivatives. However, no effects on immune cell populations, T-cell dependent antibody response, natural killer cell activity, or cytotoxic T-cell activity were observed in adult monkeys administered alirocumab or a combination of alirocumab and atorvastatin. No studies designed specifically to challenge the immune system (e.g., introduction of an infectious agent) were conducted, although no imbalances for infections were observed in general toxicity studies in monkeys or rats. Overall, these data indicate that alirocumab is unlikely to affect the immune system in adult patients.

Neurocognitive assessments: Adverse neurocognitive events (e.g., transient confusion and memory loss) have been described, primarily through patient reporting in adults on chronic statin therapy. Cholesterol and other sterols are important for nerve function in both the central and peripheral nervous system. The brain is a cholesterol-rich organ, which depends almost completely on de novo cholesterol biosynthesis for its sterols; peripheral blood lipids are unavailable to the CNS, due to blockade by the blood-brain-barrier. PCSK9 is highly expressed in brain tissues, although its function there is uncertain. Alirocumab is a 150 kDa immunoglobulin, with very low access to the brain. Therefore, it is considered unlikely that alirocumab could directly affect the structure or function of the CNS, but this leaves the possibility of effects on peripheral neurons.

No significant alirocumab-related effects on peripheral neurons were observed in toxicity studies of up to 6 months duration in rats and monkeys. The lack of neurological symptoms in animal models is reassuring.

### EXAMPLES

A middle age man had chronically elevated AST, ALT and gamma glutamyl transferase and elevated bilirubin levels and clay colored stools. There was no viral hepatitis. These enzymes and bilirubin levels returned to normal after treatment with Praluent brand alirocumab for 1-2 months and clay colored stools became normal. The patient also felt less fatigued.

In experiments with the more physiologically relevant soluble form of PCSK9, no regulation of CD81 was observed in vitro.

CD81 expression in vivo in PCSK9−/−mice and in hyperlipidemic mice expressing human PCSK9 with reduced LDLR expression (PCSK9hum/hum/LDLR+/−) were tested after administering alirocumab; no changes in total CD81 levels were observed. No effects on HCV infectivity or replication kinetics were observed with addition of extracellular PCSK9 in the presence/absence of alirocumab in vitro. Based on conflicting data, the relevant scientific literature is currently considered inadequate to conclusively address the potential impact of alirocumab on HCV infectivity. It should be noted that increased cell-surface LDLR has also been postulated to modulate HCV infectivity; statins, like alirocumab, increase cell-surface LDLR.

1. A method comprising administering a PCSK9 inhibitor to a patient who suffers from fatty liver disease or is at risk of fatty liver disease, thereby reducing the risk of fatty liver disease, severity of fatty liver disease, symptoms of such disease, or signs of such disease.

2. A method comprising administering a PCSK9 inhibitor to a patient having elevated liver enzymes to reduce said enzymes.

3. A method comprising administering a PCSK9 inhibitor to a patient having symptoms or signs of hepatic dysfunction to ameliorate said symptoms or signs and improve hepatic function.

4. A method comprising administering a PCSK9 inhibitor to a patient suffering from inflammation to ameliorate or decrease said inflammation.

5. A method comprising administering to a patient an agent to decrease lipid, fatty acid, or cholesterol synthesis, or to increase clearance of lipid, fatty acid, or cholesterol, in order to decrease risk of fatty liver disease, and decrease symptoms or signs of inflammation.

6. The method of claim 5 where said compound is selected from the group consisting of one or more of a statin, a bile acid binding resin, a fibrate, ezetitnibe (zetia), orrrega 3, ETC-1002, CETP inhibitors.

7. A method of decreasing liver damage, liver stress, or liver pathology resulting from one or more of chemical, toxic, septic, drug-induced, hypotensive, and hypoxic insults comprising administering one or more compound selected from the group consisting of a statin, a bile acid binding resin, a fibrate, ezetimihe (zetia), omega 3, ETC-1002, CEP inhibitors, and PCSK9.

8. The method of claim 7 where the liver is to be used for transplant into a patient and said compound is administered pre-harvest to the donor or post-harvest to the liver, or to the recipient, or any combination thereof.

9. The method of claim 7 where the compound is a tetracycline or an anti-tuberculosis agent.
